(19) United States
(12) Patent Application Publication
(10) Pub. No.: US 2014/0221223 A1
(43) Pub. Date: Aug. 7, 2014

(54) Use of Probes for Mass Spectrometric Identification and Resistance Determination of Microorganisms or Cells

(71) Applicant: AdvanDx, Inc., (US)

(72) Inventors: James M. Coull, Westford, MA (US); Martin Fuchs, Uxbridge, MA (US); Mark J. Fiandaca, Princeton, MA (US)

(21) Appl. No.: 13/987,316

(22) Filed: Dec. 10, 2012

(51) Int. Cl. C12Q 1/68

(52) U.S. Cl. CPC C12Q 1/6841; C12Q 1/6816
USPC 506/9; 435/6.11

(57) ABSTRACT

This invention pertains to identifying one or more hybridization probes sequestered within (or optionally released from the intact) cells or microorganisms by mass spectrometry to thereby determine a trait of the cells or microorganisms and/or to identify the cells or microorganisms themselves. The cells or microorganisms can come from a subject and the information obtained from the mass spectrometry analysis may, if clinically relevant, optionally be used to diagnose and/or treat the subject.

### INTRODUCTION

Pure colonies and liquid cultures of microorganisms can be identified using mass spectrometry (MS), particularly by use of matrix assisted laser desorption ionization—time of flight (MALDI-TOF) mass spectrometers. As a result, mass spectrometers may become a central instrument platform within microbiology laboratories. However, because accurate identification currently requires that the organisms first be isolated as pure cultures or colonies and made essentially free of contaminating media and/or other matter such as patient material (blood, etc.), there is a significant delay between when a sample is first obtained and when the accurate identification by MS can be made. Often this delay to obtain a pure isolate can be many hours or days, and in the case of clinical microbiology where rapid identification results are required to effectively treat patients (i.e., proper administration of antimicrobial drugs), such delays are associated with unsatisfactory patient outcomes, increased healthcare costs and misusage of antibiotics.

Blood culture is a standard specimen (i.e. sample) type that is commonly received for analysis in the clinical microbiology laboratory. When a blood culture turns positive, indicating that an organism is present within the culture, the culture is plated to isolate the organism as a single clonal colony in order for the MS to provide an accurate identification result. Despite efforts to process blood cultures directly to concentrate and purify microorganisms without the need for colony isolation, the accuracy is typically only in the range of 60-80%. In contrast, after clonal isolation by plating, the accuracy is in the range of greater than 95%. As such, there is a need to improve the accuracy of MS identification results directly from blood cultures.

Currently, the identification of microorganisms by MS is performed by comparing the masses (i.e. peak position and intensities that correlate with mass to charge (m/z) ratios) observed in the mass spectrum of the unknown sample to a database of mass spectra collected from known organisms. The majority of the mass peaks in these mass spectra represent the highly abundant ribosomal proteins which vary uniquely in mass between each species and genus of organism. Likely a consequence of the low occurrence of the microbe(s) of interest, there doesn't appear to be any report of the successful determination of microorganisms by the direct analysis of a blood culture using MS.

The determination of drug resistance or sensitivity is another important activity of the clinical microbiology laboratory. Typically, drug resistance and/or susceptibility of microorganisms are often determined using pure isolates in combination with phenotypic methods such microbroth dilution and disk diffusion. These properties may also be determined by use of automated phenotypic readers such as the VITEK® instrument sold by bioMerieux. In the former methods, the microorganism is exposed to a drug compound in a liquid solution and/or on a plate and the ability of the organism to grow is measured as a function of the drug presence and/or its concentration. In cases where a unique molecular mechanism is known, for example methicillin resistant Staphylococcus aureus (MRSA), genotypic or protein content can also be identified/measured to make a determination. For MRSA, the genotypic methods are often PCR-based and involve the amplification of the mecA gene, the presence of which is highly correlated to a methicillin resistant phenotype. Another molecular method is to perform fluorescence in-situ hybridization (FISH) using PNA probes directed to the mecA messenger RNA (mRNA). In the mecA PNA FISH assay a fluorescent signal demonstrates that a transcriptionally active mecA gene exists, which also correlates highly with the MRSA phenotype. The ultimate expression product responsible for the majority of MRSA is the PBP2a protein encoded by the mecA gene. Various antibody tests have been developed which utilize this protein as their target. While correlating very highly with the MRSA phenotype, the PBP2a protein cannot be detected directly from pure colonies or blood cultures with a high degree of accuracy by MS. This is most likely due to the low cellular abundance of PBP2a when compared to the ribosomal proteins. The high incidence of the ribosomal proteins makes it difficult to detect the PBP2a protein directly without additional purification steps and/or without substantially increasing the amount of sample that must be processed for the protein to be detectable.

There are additional resistance phenotypes or toxigenic phenotypes where specific proteins, genes or gene mutations are responsible for the resistance or toxigenicity phenotype of a microorganism. The genes and proteins can also be detected using antibodies or genotyping but most will likely remain refractory to determination by MS. These include, but are not limited to, the vanA and vanB gene products responsible for vancomycin resistance in enterococci, the toxin A and toxin B gene products associated with C. difficile caused diarrhea, and the carbanemase gene products (VIM, VIP, NMD, OXA, etc.) associated with carbapenem drug resistance in gram negative bacilli. Resistance and toxigenicity are also often referred to as ‘traits’ of a microorganism.

### DEFINITIONS

For the purposes of interpreting of this specification, the following definitions will apply and whenever appropriate, terms used in the singular will also include the plural and vice versa. In the event that any definition set forth below conflicts with the usage of that word in any other document, the definition set forth below shall always control for purposes of interpreting the scope and intent of this specification and its associated claims. Notwithstanding the foregoing, the scope and meaning of any document incorporated herein by reference should not be altered by the definition presented below. Rather, said incorporated document should be interpreted as it would be by the ordinary practitioner based on its content and disclosure with reference to the content of the description provided herein.

The use of “or” means “and/or” unless stated otherwise or where the use of “and/or” is clearly inappropriate. The use of “a” means “one or more” unless stated otherwise or where the use of “one or more” is clearly inappropriate. The use of “comprise,” “comprises,” “comprising” “include,” “includes,” and “including” are interchangeable and not intended to be limiting. Furthermore, where the description of one or more embodiments uses the term “comprising,” those skilled in the art would understand that in some specific instances, the embodiment or embodiments can be alternatively described using language “consisting essentially of” and/or “consisting of.”

As used herein an “aptamer” refers to a nucleic acid species that has been engineered through repeated rounds of in vitro selection or equivalently, SELEX (systematic evolution of ligands by exponential enrichment) to bind to various ‘molecular targets’ (not a nucleic acid target or target as defined below) such as small molecules, proteins, nucleic acids, and even cells, tissues or organisms.

As used herein, “nucleic acid” refers to a polynucleobase strand formed from nucleotide subunits composed of a nucleobase, a ribose or 2′-deoxyribose sugar and a phosphate group. Some examples of nucleic acid are DNA and RNA.

As used herein “nucleic acid analog” refers to a polynucleobase strand formed from subunits wherein the subunits comprise a nucleobase and a sugar moiety that is not ribose or 2′-deoxyribose and/or a linkage (between the sugar units) that is not a phosphate group. A non-limiting example of a nucleic acid analog is a locked nucleic acid (LNA: See for example, U.S. Pat. Nos. 6,043,060, 7,053,199, 7,217,805 and 7,427,672). See: Janson and During, “Peptide Nucleic Acids, Morpholinos and Related Antisense Biomolecules”, Chapter 7, “Chemistry of Locked Nucleic Acids (LNA)”, Springer Science & Business, 2006 for a summary of the chemistry of LNA.

As used herein the phrase “nucleic acid mimic” refers to a nucleobase containing polymer formed from subunits that comprise a nucleobase and a backbone structure that is not a sugar moiety (or that comprises a sugar moiety) but that can nevertheless sequence specifically bind to a nucleic acid. An example of a nucleic acid mimic is peptide nucleic acid (PNA: See for example, 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, WO92/20702 and WO92/20703). Another example of a nucleic acid mimic is a morpholino oligomer. (See Janson and During, “Peptide Nucleic Acids, Morpholinos and Related Antisense Biomolecules”, Chapter 6, “Morpholinos and PNAs Compared”, Springer Science & Business, 2006 for a discussion of the differences between PNAs and morpholinos.

It is to be understood that the scope of this invention is not limited the use of “traditional” aminoethyl glycine-based PNA probes. The PNA probes include all possible PNA backbone configurations. As used herein, “peptide nucleic acid” or “PNA” refers to any oligomer or polymer comprising two or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference. The term “peptide nucleic acid” or “PNA” can also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

As used herein “nucleobase” refers to those naturally occurring and those non-naturally occurring heterocyclic moieties commonly used to generate polynucleobase strands that can sequence specifically bind to nucleic acids. Non-limiting examples of nucleobases include: adenine (“A”), cytosine (“C”), guanine (“G”), thymine (“T”), uracil (“U”), 5-propynyl-uracil, 2-thio-5-propynyl-uracil, 5-methylcytosine, pseudoisocytosine, 2-thiouracil, 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

As used herein “nucleobase sequence” refers to any nucleobase containing segment of a polynucleobase strand (e.g. a subsection of a polynucleobase strand). Non-limiting examples of suitable polynucleobase strands include oligodeoxynucleotides (e.g. DNA), oligoribonucleotides (e.g. RNA), peptide nucleic acids (PNA), PNA chimeras, nucleic acid analogs and/or nucleic acid mimics.

As used herein “nucleobase containing subunit” refers to a subunit of a polynucleobase strand that comprises a nucleobase. For oligonucleotides, the nucleobase containing subunit is a nucleotide. For other types of polynucleobase strands (e.g. nucleic acid analogs), the nucleobase containing subunit will be determined by the nature of the nucleobase containing subunits that make up said polynucleobase strand (i.e. a polynucleobase polymer).

As used herein “polynucleobase strand” refers to a complete single polymer strand comprising nucleobase containing subunits.

As used herein, “sequence specifically” refers to hybridization by base-pairing through hydrogen bonding. Non-limiting examples of standard base pairing include adenine base pairing with thymine or uracil and guanine base pairing with cytosine. Other non-limiting examples of base-pairing motifs include, but are not limited to: adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); thymine or uracil base pairing with any of: 2-aminopurine, N9-(2-amino-6-chloropurine) or N9-(2,6-diaminopurine); and N8-(7-deaza-8-aza-adenine), being a universal base, base-pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine) or N9-(7-deaza-8-aza-guanine) (See: Seela et al., Nucl. Acids, Res.: 28(17): 3224-3232 (2000)). It is to be understood however that a probe or primer can hybridize with sequence specificity even in the presence of one or more point mutations, insertions or deletions such that the remaining complementary nucleobases are able to base-pair.

Probes are typically used under suitable hybridization conditions. The extent and stringency of hybridization is controlled by a number of factors well known to those of ordinary skill in the art. These factors include the concentration of chemical denaturants such as formamide, ionic strength, detergent concentration, pH, the presence or absence of chaotropic agents, temperature, the concentrations of the probe(s) and quencher(s) and the time duration of the hybridization reaction. Suitable hybridization conditions can be experimentally determined by examining the effect of each of these factors on the extent and stringency of the hybridization reaction until conditions providing the required extent and stringency are found. When properly applied, suitable hybridization conditions result in sequence specific hybridization of a probe to its complementary target.

As used herein “target” or “target sequence” refers to a nucleobase sequence (often a subsequence of the entire molecule) of a polynucleobase strand sought to be determined.

### DISCLOSURE OF THE INVENTION

It should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

It is an advantage that embodiments of this invention permit MS to be used to perform accurate identification of microorganisms without the need to first isolate a pure colony or broth culture.

It is an advantage that embodiments of this invention permit MS to be used to perform accurate determination of antimicrobial resistance, susceptibility, toxigenicity or other attributes of microorganisms that cannot presently be determined by MS.

Embodiments of this invention use probes (e.g. 12 to 20-mers in length). A probe can be any probe that can sequence specifically hybridize to rRNA, mRNA, and/or plasmid and genomic DNA target sequences of intact microorganisms. Suitable probe types include, but are not limited to, nucleic acids, nucleic acid analogs and nucleic acid mimics. The nucleobase sequence of each probe is selected to hybridize to a target sequence that if present in the microorganism will correlate with a condition of interest (e.g. will identify the microorganism or a trait of the organism). Generally PNA probes are used. The probes are permitted to hybridize (the “hybridization step”) to nucleic acid targets within the intact microorganisms in a sample of interest using hybridization techniques known and routinely used in the art (for example techniques used in FISH analysis).

In some embodiments, the probes can be aptamers or other specific binding agents of a known mass that are capable of selectively/specifically binding to a particular target within a microorganism(s) that may be present in the sample. For simplicity, the binding of the aptamer or other specific binding agent (also for simplicity these will be referred to herein as a “probe”, “probes”, “hybridization probe” or “hybridization probes” depending on whether the singular or plural is required) to its molecular target is likewise referred to herein as a “hybridization step”.

Following the hybridization step, the excess probe (or probes in the case of a probe mixture) is removed by washing (the “wash step” or “washing step”) the cells/microorganisms (i.e. the ‘washing step’ removes substantially all excess unbound/unhybridized hybridization probe from the microorganisms). Consequently, substantially all hybridization probes remaining within the intact cells/microorganisms after the washing step or steps will be specifically hybridized to their respective target molecule(s) (rRNA, mRNA or DNA) within the cell. If the target is not present within the cell, the hybridization probe will have been substantially washed away from the sample (i.e. not be present within the microorganisms) and will not be available for detection in subsequent MS analysis.

The intact microorganisms/cells of the sample can then be lysed (the “cell lysis step”; typically performed with formic acid (or another volatile composition that is capable of cell lysis and is compatible with MS analysis) because it is compatible with MS analysis) to release the cell contents, including the cellular ribosomal proteins as well as any hybridization probes still contained therein for analysis by MS. The cell lysis process may also be accomplished using standard MS sample preparation methods or modifications thereof. The product of the lysis reaction can then be analyzed by MS (the “MS analysis step”) such as by MALDI-TOF MS using routine procedures.

The result of MS analysis step is a mass spectrum that comprises of the masses of nucleic acids, proteins and the hybridization probe(s) that were present in the microorganisms when lysed. If a rRNA-directed hybridization probe was used (in the hybridization step) for species determination, ample hybridization probe should be available for analysis (For example, each ribosome consists of two subunits, each subunit containing RNA and multiple proteins (in prokaryotes, the small subunit is composed of 16S rRNA and 21 proteins and the large subunit is composed of 5S and 23S rRNA and 31 proteins; in eukaryotes the small subunit is composed of 18S rRNA and 33 proteins while the large subunit is composed of 5S, 28S and 5.8S rRNA and 46 proteins). The rRNA-directed probes are generally designed to target a particular rRNA (e.g. the 16S rRNA in prokaryotes). Thus, there may be approximately one molecule of probe for every ribosome within the cell following hybridization if a single rRNA-directed probe is used in the hybridization step. It is also not without significance that; 1) the hybridization probes possess a small molecular weight relative to the ribosomal proteins; 2) in the case of PNA probes, the hybridization probes are known to inherently “fly” well in the MS without the nucleobase loss; 3) the hybridization probes can optionally be chemically tagged to enhance their detection by the MS instrument; and/or 4) the hybridization probes may be easily discernible in the mass spectrum and may even dominate the spectrum as compared to ribosomal proteins.

Consequently, one embodiment of this invention pertains to a method comprising: 1) contacting microorganisms or cells of a sample with one or more hybridization probes capable of determining a condition of interest within said microorganisms or cells for a period of time sufficient for said hybridization probes to sequence specifically hybridize to their respective target sequences associated with said condition of interest within said microorganisms or cells; 2) washing said microorganisms or cells to remove excess hybridization probes; 3) lysing said microorganisms or cells to produce a cell lysate; and 4) analyzing said cell lysate by MS to identify one or more hybridization probes contained in said cell lysate. Said method can further comprise, determining one or more conditions of interest associated with said microorganisms or cells where said one or more conditions of interest correlate with the identity of said one or more hybridization probes identified in step 4) of the method. Said method can further comprise making a diagnostic determination based upon the conditions of interest so determined. Said method can further comprise making a treatment recommendation for a subject from whom the sample was obtained based upon said diagnostic determination.

Those of skill in the art will appreciate that the period of time sufficient for the hybridization probes to sequence specifically hybridize to their respective target sequences is largely dependent on the probe type, the concentration of the hybridization probes in the solution and the hybridization conditions (e.g. the salt concentration, pH and temperature are very important variables). For example, PNA probes will often hybridize sufficiently in 5-30 minutes whereas nucleic acid probes and nucleic acid analog probes may take from 30 minutes to 2 hours or more to sufficiently hybridize to their respective target sequences.

According to some embodiments of the method, a hybridization probe can be designed chemically so that independent of its nucleobase sequence the mass of the probe is unique, while at the same time the probe sequence can be designed so as to be specific for a rRNA target of a particular species or genus of microorganism sought to be determined. Therefore, the presence of a particular unique probe mass within the mass spectrum of a sample that has been hybridized with hybridization probe and washed to remove excess and unbound hybridization probe will be diagnostic for the presence of the organism within the sample. The increased sensitivity of the spectrometer for said hybridization probes (as opposed to the ribosomal proteins) should permit the direct analysis of complex samples, such as blood cultures, without the need to first isolate a pure colony.

In practice, since one does not, a priori, know the identity of the organism in a blood culture then one may contact the cells/microorganism in the blood culture sample with a mixture of probes wherein each probe possesses a unique mass corresponding to a unique sequence for a condition of interest that may exist within the blood culture (i.e. the probes of the probe mixture may be selected to determine, for example, what cells/microorganism(s) is/are in the blood sample and/or what traits do cells and/or microorganisms of the blood culture possess). Only the hybridization probe or probes corresponding to the organism(s) actually present in the blood culture will be observed in the mass spectrum since other “non-binding” probes will be removed in the wash step. In the case of a mixed blood culture where more than one species is present, if the probes of the probe mixture are judiciously selected to determine different species, then a corresponding number of mass peaks for the probes specific for each species can be observed in the mass spectrum analysis.

For example, current blood sample analysis typically involves approximately 10 different “organism identifications” (and by extension approximately ten probes or probe sets could be used to analyze the majority (70% to 90%) of species (i.e. conditions of interest)) that are commonly required to be analyzed from positive blood cultures. Consequently, a rather simple probe set would could be created and used in a MS-based assay according to embodiments of this invention that would be capable of identifying the majority of conditions of interest commonly determined for positive blood cultures. It is to be understood that in some embodiments, it may be desirable that some “organism identifications” using hybridization probes be made to the species level, whereas other “organism identifications” be made to a class of phenotypically or therapeutically similar species such as the coagulase negative staphylococci. Current methodologies routinely used in the art (e.g. sequence alignments and other standard probe design tools) permit the design of hybridization probes that can be uniquely tailored for the determination of each particular condition of interest. Many useful probes sequences are already known and routinely used in the art. The hybridization probes can typically be custom synthesized by commercial vendors and then be mixed to prepare a probe mixture that can be used to simultaneously determine all possible conditions of interest in a single MS analysis.

In some embodiments, determination of resistance can be performed using probe-based MS identification wherein the hybridization probes are selected to bind to specific genes or gene transcripts instead of, for example, rRNA. For example, it is known that MRSA, upon exposure to methicillin type drugs such as oxacillin, will increase the production of mecA mRNA and PBP2a protein within the microorganism as a consequence of the presence of the mecA gene. This can be visually observed by PNA FISH using a mixture of fluorescently labeled PNA probes which hybridize to different sequences within the mecA mRNA. To convert this FISH-based assay to an MS-based assay one needs only to, for example, adjust the mass of each PNA probe through attachment of chemical tags (e.g., N- or C-terminal amino acids which do not impact the hybridization) such that each probe then has the same molecular weight. Consequently, if, 1) the probes are mixed; 2) the sample (e.g. a blood culture) is contacted with the probes and said probes are allowed to hybridize to their respective target sequences; 3) excess probes are removed using a wash step; 3) the cells/microorganisms of the blood culture are lysed; and 4) the lysate is analyzed by MS, then it should be possible to determine if MRSA is present in the blood culture. Specifically, if the mecA mRNA is present within the cells a peak should be present in the mass spectrum that corresponds to the adjusted mass of the probes in the mixture. Thus many different probes can contribute to the unique mass peak in the mass spectrum that is diagnostic for a particular condition of interest.

It is to be understood that if there is a highly expressed gene producing many copies of mRNA associated with a condition of interest only one probe may be required to provide sufficient MS-sensitivity whereas if it is a very low expression level of target mRNA sequence associated with a particular condition of interest, many probes may be needed and the mass of the many probes can be mass-adjusted so that they all comprise the same mass. In this way the intensity of the many different probes are additive and produce a proportionally larger signal in the MS spectrum. One can also produce a multiplex mixture of probes whereby the mixture contains different sets of probes where each set of probes is specific for a particular condition of interest and each individual probe of a specific set is mass-adjusted to possess the same mass and wherein the probes for each different condition of interest possess a unique mass as compared with the probes for all other conditions of interest sought to be determined using the multiplex mixture.

It is to be understood that embodiments of this invention permit one to target both rRNA and mRNA in the same assay. For example, a probe of unique mass can be designed to specifically hybridize to rRNA that is characteristic for S. aureus and it can be combined with a probe set that specifically hybridizes to mRNA associated with the presence of the mecA gene (that can be used to identify the trait of methicillin resistance) wherein the probes of the probe set comprise a unique mass as compared with the probe that specifically hybridizes to the rRNA of S. aureus. Thus, when both masses are observed in the MS spectrum, the sample can be said to contain MRSA. In some embodiments, further multiplexing of the assay can be achieved by, for example, adding an additional rRNA-directed probe for coagulase negative staphylococci to (CNS), wherein said rRNA-directed probe for coagulase negative staphylococci comprised still another unique mass as compared with the mass of any other probes of the mixture. In that way, the MS analysis can be used to distinguish S. aureus, from CNS from, MRSA and from MR-CNS.

The relative area of the mass peaks may provide additional important information. For example a relatively large mecA probe peak relative to the S. aureus rRNA probe peak may indicate a highly expressing MRSA whereas a small mecA probe peak relative to the rRNA probe peak may indicate a weakly expressing MRSA. Such information could be used to better diagnose patient conditions as well as select the amounts and types of antibiotic treatments administered to patients.

Certain traits within microorganisms are encoded on extrachromosomal plasmids within a microorganisms. For example the carbapenenmase NDM-1 which confers resistance to certain carapenem drugs is often found encoded on a plasmid with the bacterium Klebsiella pneumonieae. Often the plasmid is present in many copies and while it will be possible to detect the mRNA expressed from the NDM-1 gene in the plasmid, it may further be possible to directly detect the gene by hybridization of a NDM-1 specific probe set to the DNA sequence of the NDM-1 gene. The ability to directly detect the NDM-1 gene may obviate the need to induce the expression of the gene (for example, by exposure of the microorganism to a drug such as a caebapenem) for the purpose of detecting its mRNA expression product, thereby resulting in a simplified assay. Likewise, if the sensitivity of the MS analyzer is quite good and/or the probes are tagged or chemically modified so as to make them very detectable by the spectrometer, then one may directly detect the presence of a single copy chromosomally encoded gene by using the aforementioned probe set where each member of the set is adjusted to the same mass and thereby contributes to the observed mass peak in the mass spectrum.

In the foregoing discussion, cells/microorganism were lysed and processed such that any hybridized probe can be liberated with the ribosomal proteins and both would be available for MS analysis. For typical MS microbial sample preparation, the cells and their contents can be dissolved using a strong (volatile) acid solution such as 70% formic acid in combination with a solvent such as acetonitrile. Rather than lysing the cells/microorganisms and liberating their contents, in some embodiments it may be advantageous to isolate the hybridization probes from the intact cells/microorganisms following the hybridization step and the washing step. By isolating the hybridization probes from the intact cells, it may be possible to further increase the sensitivity of the assay and/or to further multiplex the assay. This is because the isolation of the hybridization probes from the intact cells/microorganisms will eliminate much of the cellular debris (e.g., ribosomal and other proteins) that would otherwise generate background in the MS spectrum. A cleaner MS spectrum should permit increased sensitivity of the assay.

For example, once the cells/microorganisms have undergone the hybridization step and the wash step, any bound probes can be removed from the intact cells/microorganisms by, for example, heat treatment that denatures the hybridization probes from their respective target(s). In some embodiments, solvents, detergents, RNAses or combinations thereof (with or without added heat) can also be used to cause dissociation and dissolution of the hybridization probes without causing lysis of the cells/microorganisms or elution a majority of the cellular contents. Because the hybridization probes are relatively small in size, they will easily pass though the cell membrane and into the solution once dissociated from their respective target sequences. Consequently, the resulting recovered probe solution can then be analyzed by MS. Because the resulting recovered probe solution is less complex, the sensitivity of the MS detector will be increased, as will the mass resolution. This means that assays where the hybridization probes are recovered from the intact cells/microorganisms should exhibit superior sensitivity and resolution as compared to embodiments where cells/microorganisms are lysed. Consequently it is expected that such assays will provide more diagnostic information than if the ribosomal proteins and other cellular materials are present.

Consequently, one embodiment of this invention pertains to a method comprising: 1) contacting microorganisms or cells of a sample with one or more hybridization probes capable of determining a condition of interest within said microorganisms or cells for a period of time sufficient for said hybridization probes to sequence specifically hybridize to their respective target sequences and thereby produce a probe/target complexes associated with said condition of interest within said microorganisms or cells; 2) washing said microorganisms or cells to remove excess hybridization probes; 3) treating said microorganisms or cells with heat and/or other denaturing conditions for a period of time sufficient to thereby cause said probe/target complexes to denature and the denatured probes to diffuse outside of the intact cells/microorganisms; 4) recovering said denatured probes that exist outside of said intact cells/microorganisms; and 5) analyzing said recovered probes by MS to identify one or more of said recovered probes. Said method can further comprise, determining one or more conditions of interest associated with said microorganisms or cells where said one or more conditions of interest correlate with the identity of said one or more recovered probes identified in step 5) of the method. Said method can further comprise making a diagnostic determination based upon the conditions of interest so determined. Said method can further comprise making a treatment recommendation for a subject from whom the sample was obtained based upon said diagnostic determination.

Those of skill in the art will appreciate that a period of time sufficient to cause a probe/target complex to denature and for the denatured probes to diffuse outside of the intact cells/microorganisms will be highly condition dependent. For example if only heat is used, the process will be slower than if heat is combined with chemical denaturants (e.g. formamide). Similarly, the process will be slower if only chemical denaturants are used at ambient temperature (as compared with elevated temperature—e.g. 35-80° C.). Generally, this ‘denaturing and diffusion step’ can be accomplished from between 10 minutes to 2 hours.

Probes released from the cells by heating can be subsequently captured/concentrated recovery of liquid surrounding them during the treatment by heat and/or denaturing conditions. For example, this will be accomplished by pelleting the cells. The recovered supernatant can be analyzed directly or concentrated if necessary. Alternatively, recovered probes present in the supernatant can be collected by binding to complementary sequences immobilized on beads or other surfaces such as slides. Such a capture/concentration step can facilitate their introduction into the mass spectrometer and allow further washing to remove cellular contents or other potentially interfering agents or concentration of the probes into a small volume. In some embodiments the probes can be collected from the beads. In some embodiments, the beads can be directly analyzed in the MS instrument.

The current requirement upon the instrumentation to resolve the ribosomal proteins in a complex sample often pushes the sensitivity and resolution power of existing technology to their limits, especially for certain sample types such as blood cultures. The hybridization probes discussed herein fit well into the “sweet spot” of any given MS platform. Similarly, the masses of the hybridization probes may be adjusted to place them into an available “mass window” which may be available for a particular sample-type. Mass windows are areas of a mass spectrum where there are few or no mass peaks present. This approach could enable samples that are currently undetectable due to the presence of substances which interfere with the ribosomal proteins needed to microbe determinations. Examples of sample types where it may be useful to employ probes tailored to mass windows include, but are not limited to, blood cultures, whole blood, blood products, platelet preparations, stool, urine, and pulmonary secretions. Furthermore this approach allows for less sample manipulation, thereby simplifying and perhaps increasing the sensitivity of the MS method for detection of microorganisms in certain sample types.

When using embodiments of this invention, the MS instrument and its corresponding software and results database may not need to be as complex as currently in use because the mass spectra of the invention may be less complex due to the intensity of the probe peaks and relative absence of peaks corresponding, ribosomal and other proteins as well as other cellular debris.

The currently employed method of microorganism detection using ribosomal proteins requires establishment and maintenance of a database of mass spectrographs to which any new data is compared (the “natural spectra+database analysis approach”). An algorithm is used to compare the sample mass trace to the database to derive the identification of the new sample. Not only does this approach require frequent maintenance of the database, it may also require a separate database for every sample type (blood, stool, etc.). Because the unique probe masses observed in the mass spectrum represent defined compounds that are present because they were added to the sample and are not natural compounds (i.e., proteins) they are not subject to mutation, natural variation, evolution or other change which could confound results and which require frequent updating of the databases in the currently practiced methods. Additionally, the detection of hybridization probes that are specifically added and then detected in the MS trace allows the same database to be used across different samples types since masses that correlate with materials present in a particular sample type are generally of no concern.

Another pitfall of the current “natural spectra+database analysis approach”, which has been documented in the literature, is the frequent inability to resolve multi-organism (i.e., multiple species) mixtures since the algorithms are often not able resolve spectra comprised of mass peaks from multiple organisms. Furthermore, to improve the certainty of a result a probe of unique sequence may be doubly “tagged” such that a given unique hybridization probe sequence produces two peaks in the mass spectrum, in this way a result where both peaks are observed for the same probe will be of higher confidence. We envision using internal control probes to correct for various sample handling steps or to improve quantitation. For example, internal control probes may help to detect mismatch hybrids if they occur, such that a control probe giving a 1× signal compared to a specific hybridization probe on the same target providing a 0.5× signal may indicate a mismatched hybrid (e.g. point mutation or a heterogeneous genotype).

The concept of double or multiple labeling may be further applied to maximize the amount of information derived from a particular sample (e.g. blood culture). Multiplex probe mixtures may include several probes which universally detect various groupings of microorganisms. The various groupings may include probes that are specific for various phylogenetic or phenotypic classes. Groupings may include, but are not limited to, bacteria, fungi, gram-positive bacteria, gram-negative bacteria, Candida genus, Enterobacteriaceae, Acinetobacter genus, coagulase negative staphylococci, or non-E. faecalis enterococci. Other groupings by Genus, Family, Order, Class, Kingdom, Phylum or other phylogenetic distinction are within the scope of the present invention.

As stated above, one may employ a strategy of doubly detecting many organisms or classes of organisms to improve the certainty of results or for other purposes. For example, one could design a multiplex probe set that ensures that vast majority of microorganisms present in a sample are detected with at least one probe; for instance a universal bacterial probe. This could act as a positive control for the assay.

It is also within the scope of this invention to provide very specific probe sets not necessarily to detect specific organisms in a sample (e.g. stool) but to detect or estimate total bacterial load as a means to diagnosis of a condition of interest in a patient. An example of a suitable probe set might be one that is designed as a multiplex probe set that is capable of detecting several higher order classes of targets, for example, Enterobacteriaceae (family), Firmicutes (phylum), Bacilli (class) and Clostridia (class). Use of this probe set in the method embodiments of this invention could be used to get a snapshot of the total bacterial load and composition from a sample, such as for example, stool. Another example would be the use of a universal bacterial probe to directly and rapidly measure the bacterial load in a blood product such as a platelet preparation just prior to administration of the platelets to the patient. Current blood culture and respiratory methods are slow meaning increasing the risk that patients receive bacterially contaminated platelets because bacteria have grown to harmful levels during the time between when the platelets were sampled and the test results are available. Thus, even though a blood culture or respiratory test may show no, or low, level contamination, the actual contamination load in the platelets may be quite high when the patient is infused with them.

Although the selection of certain probe sets may be sample dependent, it is also within the scope of this invention to use the same probe set across various sample types. Specific detection of a particular organism of interest, for instance S. aureus, could be performed using the same probe or probe mixture regardless of the sample type. Where probes are released from intact cells prior to analysis, the resulting MS trace is not likely to differ across sample types. So not only can the same kit be used across different sample types, but the same data analysis may be uses as well.

Because the methods employed by this invention do not require the comparison of obtained spectra to the spectra of known organisms to make a determination, it is an advantage of this invention that the computing power and user interface requirements of the associated MS analysis will likely be greatly minimized as compared to the current methodologies. Likewise, because certain probe types (e.g. PNAs) inherently “fly” well in a MS, we expect that MS hardware requirements could possibly be relaxed, for example, in terms of the laser strength, power usage, vacuum tube length, cost, etc. Because MS hardware is typically very costly, we expect that these relaxed requirements may result in the ability to use less expensive and perhaps smaller instruments than those currently used. Where specific masses are expected from a sample, and the number of possible specific probe masses to be determine is limited to a small number (for example, 10 distinct possibilities) it is easy to conceive of an instrument that could automatically “call” the result with high confidence using very basic software.

It is to be understood that the MS analysis is not limited to utilizing MALDI mass spectrometers but it may be used with any type of mass spectrometer that is able to detect the hybridization probes from the samples. For example, instead of MALDI interface electrospray or other interface may be used. Furthermore instead of a TOF, the mass analyzer could be a quadrupole, ion-trap or other ion separation modality. Virtually any ion source or ionization technique capable of introducing a probe into the MS platform may be used and the ion-separation and detection modes may be any that can be, or are typically used to detect probes such as PNA, oligonucleotides, peptides, and their analogues.

It is to be understood that embodiments of the methods disclosed herein could be used for a variety of non-medical uses such as pharmaceutical production, manufacturing, waste water analysis, food analysis, agriculture, veterinary diagnostics and industrial hygiene.

Another advantage of the present invention is the ability to “kit” a discreet set of probes that could be easily validated for a specific determination (e.g. MRSA analysis). The current use of MS which asks the broad question “what is in the sample” may be difficult to validate, since all possible answers have to be checked. A simplified, kitted, use of the technology asks the question “is this (analyte) in the sample”, where the number of possible analytes may be as few as one. This type of question is a much easier answer to validate for regulatory purposes. Thus, it is an advantage that the method embodiments of this invention may prove to be superior with respect to clinical validation/regulatory approval.

When it is desirable to recover probes from intact cells for analysis by MS, so-called “extraction handles” can be added to the probes as a way to selectively release the probes from the sample through differential solubility of the PNA from the sample in general. Extraction handles could be envisioned to allow PNA to preferentially dissolve in organic solvents such as methanol or water or lipids such as mineral oil. In short, since the probe is being added to the sample it may be preferentially labeled so that it is easy to extract at the end of processing. For example, the probe could be made methanol soluble so that it could be extracted from the sample without also dissolving the cells or other cellular materials. The probes may also be tagged with an affinity handle to further recover, localize, purify or concentrate them prior to analysis.

### PROPHETIC EXAMPLES

Aspects of the present teachings can be further understood in light of the following examples, which should not be construed as limiting the scope of the present teachings in any way.

### Example 1

### Preparation of Microorganisms from a Pure Isolate

Colonies are prepared on an agar plate containing media sufficient to support growth of microorganisms of interest. After a sufficient growth period at a sufficient growth temperature, one to three colonies of microorganism are harvested and suspended in 0.3 milliliters of deionized water. Nine hundred microliters of 100% ethanol are added; the mixture is mixed by inversion, and then centrifuged at 12,000×g for 3 minutes. The supernatant is decanted, the sample is centrifuged a second time, any remaining supernatant is carefully removed and the pellet is air dried.

### Example 2

### Preparation of Microorganisms from a Blood Culture

One milliliter of a positive blood culture is added to 0.2 milliliters of a 5% saponin solution, then votexed thoroughly to mix. After 5 minutes of incubation at room temperature, the tube is centrifuged at 16,600×g for 1 minute. The supernatant is decanted. The pellet is washed with 1 milliliter of deionized water, and re-centrifuged at 16,000×g for 1 minute. The supernatant is decanted, and the pellet is air dried.

### Example 3

### Viability Test of Prepared Microorganisms

The pellet produced from either Example 1 or Example 2 is resuspended in 0.1 milliliter of deionized water. 10 microliter of the suspension is used to inoculate either an agar plate or a liquid culture containing media sufficient to support growth of microorganisms. After a sufficient growth period at a sufficient growth temperature, either colonies are produced on the agar plate or the liquid culture has become turbid.

### Example 4

### Hybridization of PNA in Solution

The pellet produced from either Example 1 or Example 2 is resuspended in 20 microliter of deionized water. To the mixture is added 0.2 milliliter of PNA reagent (0.025 M Tris-HCl; 0.1 M NaCl; 50% (v/v) Methanol; 0.1% Sodium Dodecyl; 0.5% Yeast Extract Solution; 25-250 nM and one or more PNA probes, the nucleobase sequence of which is selected to determine a condition of interest). The contents are mixed by vortexing and the samples are incubated at 55° C. for 30 minutes. After 5 minute centrifugation at 10,000×g, the supernatant is removed and the pellet is resuspended in 0.5 milliliter of Wash Buffer (0.005 M Tris-HCl pH 9.0, 0.025 M NaCl and 0.1% Triton X-100). Samples are incubated at 55° C. for 10 minutes, re-pelleted, and re-suspended in 0.5 milliliter of Wash Buffer and heated at 55° C. for 10 minutes.

### Example 5

### Hybridization of PNA on a Solid Support

The pellet produced from either Example 1 or Example 2 is resuspended in 0.1 milliliter of deionized water. 10 microliter of sample and 1 drop of AdvanDx PNA FISH Fixation Solution (AdvanDx product No: CP0021) are mixed in a well on the surface of the solid support. The sample is fixed by placing it at 55° C. for 20 min, then in 96% (v/v) ethanol for 5 minutes, then air dried. Hybridization is performed by adding 1 drop of a PNA FISH hybridization solution (such as S. aureus PNA FISH, KT001, AdvanDx Woburn, Mass.) and a cover slip, then incubating at 55° C. for 30 minutes. The coverslip is removed, and the sample is washed for 30 minutes at 55° C. in 1×PNA FISH Wash Solution. Optionally, the wash step is repeated, and the sample is air dried.

### Example 6

### Detection of Bound PNA by Mass Spectrometry

The method provides a means to detect PNA bound in a previous hybridization step to be detected by first dissolving the detected microorganisms in a solvent.

The solution produced from Example 4 is pelleted by 5 minute centrifugation at 10,000×g. Between five and fifty microliters of 70% formic acid is added to the pellet dependent on the pellet size, followed by an equal volume of acetonitrile. The sample is centrifuged again at 12,000 g for 3 minutes. 0.5 to 5.0 microliters of the supernatant are spotted on a solid surface and air dried. The sample is overlaid with matrix (saturated α cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition of interest associated therewith.

### Example 7

### Detection of Released PNA by Mass Spectrometry

The method provides a means to detect PNA bound in a previous hybridization step to be detected by releasing the PNA into a solvent.

The solution produced from Example 4 is pelleted by 5 minute centrifugation at 10,000×g. 10 to 100 microliters of 1M ammonia in methanol is added to the pellet, votexed, then incubated at 40° C. for 10 to 20 minutes to release the PNA from the microorganisms. The sample is centrifuged at 12,000×g for 3 minutes. 0.5 to 5.0 microliters of the supernatant is spotted on a solid surface and air dried. The sample is overlaid with matrix (saturated α cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition of interest associated therewith.

### Example 8

### Determination of Resistance by Detection of Bound PNA by Mass Spectrometry

An aliquot of a blood culture is combined with a solution containing an antibiotic of interest. The combined solutions are incubated to allow exposure of the organisms in the culture to the drug for a period and at a temperature sufficient to stimulate a physiological reaction to the drug. One milliliter of the blood culture mixture is added to 0.2 milliliters of a 5% saponin solution, then vortexed thoroughly to mix. After 5 minutes incubation at room temperature, the tube is centrifuged at 16,600×g for 1 minute. The supernatant is decanted. The pellet is washed with 1 milliliter of deionized water, and re-centrifuged at 16,000×g for 1 minute. The supernatant is decanted, and the pellet is resuspended in 20 microliter of deionized water. To the mixture is added 0.2 milliliter of PNA reagent (0.025 M Tris-HCl; 0.1 M NaCl; 50% (v/v) Methanol; 0.1% Sodium Dodecyl; 0.5% Yeast Extract Solution; 25-250 nM and one or more PNA probes). One or more of the PNA probes may be complementary to rRNA sequences (for identification of the organism). One or more of the PNA probes may be complementary to the mRNA of a resistance gene. If more than one probe is used for identification of the resistance gene it is preferred to design the probes such that some or all of them have the same mass. The contents are mixed by vortexing and the samples are incubated at 55° C. for 30 minutes. After 5 minute centrifugation at 10,000×g, the supernatant is removed and the pellet is resuspended in 0.5 milliliter of Wash Buffer (0.005 M Tris-HCl pH 9.0, 0.025 M NaCl and 0.1% Triton X-100). Samples are incubated at 55° C. for 10 minutes, re-pelleted, and re-suspended in 0.5 milliliter of Wash Buffer and heated at 55° C. for 10 minutes. The solution is pelleted by 5 minute centrifugation at 10,000×g. Between five and fifty microliters of 70% formic acid is added to the pellet dependent on the pellet size, followed by an equal volume of acetonitrile. The sample is centrifuged again at 12,000 g for 3 minutes. 0.5 to 5.0 microliters of the supernatant are spotted on a solid surface and air dried. The sample is overlaid with matrix (saturated α cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition(s) of interest associated therewith.

### Example 9

### Analyzing Microorganisms from Low-Titer Samples

In many cases, it is of interest to analyze microorganisms from samples in which they are low in number, such as blood or water. It is possible to concentrate the microorganisms by filtering such samples through a membrane filter having a pore size small enough to retain the bacteria of interest. For blood, such filtration requires that the blood cells first be lysed and the resulting cell debris treated to solubilize it. Selective lysis of the blood using saponin and high-frequency ultrasound accomplishes this requirement.

Lysis solution is prepared by adding 115 mg of saponin to 10 mL of 0.1M sodium phosphate buffer, pH 8 and vortexing to dissolve. 11.25 Units/mL of proteinase are added and vortexed briefly to dissolve. The solution is filtered using a 0.2 μm, 32 mm, PES syringe filter.

Blood samples are prepared by adding 1 mL of lysis solution and 1 mL of blood to a 3 mL, round bottom, glass Covaris tube. The samples are mixed by inversion.

The bath on a Covaris S2 Sonicator is filled with deionized water, heated to 37° C., and degassed for 30 minutes. The tubes are loaded into the custom tube holder designed to fix the X and Y axis. The samples are warmed and mixed for 100 seconds at an intensity of 1, 10% duty cycle, and 1000 cycles per burst. Then the intensity is increased to 2 for 60 seconds. Finally, the cycles per burst is decreased to 200 for 60 seconds.

The lysate is concentrated on a metal coated polycarbonate track etched membrane (PCTE) filter with a pore size of 0.6 microns. The metal can be gold or other suitable metal.

### Concentration Method

Filter entire lysate using a vacuum equivalent 5 to 15 inches of Hg. Rinse filter and holder 3 times with 830 μL each of 1×PBS while vacuuming. Turn off and purge vacuum.

### Optional Growth Step

Place the membrane filter onto an agar plate (composition chosen to be suitable for the microorganisms of interest). Incubate at 37° C. for 2-6 hours to allow growth of microcolonies.

### Optional Hybridization and Wash Steps

Place the membrane filter into a thermostatted holder fitted with a disposable plastic tube that allows fluid to be dispensed onto the membrane. Filter PNA FISH Flow Hybridization Buffer immediately prior to use with a 13 mm, 0.2 μm, polytetrafluorethylene (PTFE) syringe filter. Add 400 μL of filtered or PNA FISH Flow Hybridization Buffer containing 100 nM to 500 nM or 50 nM probe for bacteria or yeast respectively to the holder. Cover the holder to prevent evaporation. Heat the retentate and hybridization buffer in the holder for 30 minutes at 55° C. Vacuum away hybridization buffer. Turn off and purge vacuum. Add 500 μL of PNA FISH Flow Wash Buffer to the holder. Cover holder to prevent evaporation. Heat the retentate and wash buffer in the holder for 10 minutes at 55° C. Vacuum away wash buffer. Turn off and purge vacuum. Optionally repeat steps.

### Analysis

Dry the membrane filter with trapped (optionally hybridized and washed) microorganisms (optionally microcolonies). The membrane is overlaid with matrix (for example saturated α cyano-4-hydroxycinnamic acid, 50% acetonitrile, 2.5% trifluoroacetic acid) and air dried. The membrane is placed on the MALDI sample plate and held in place using a metal ring that establishes a conductive path from the metal coating on the membrane to the sample plate. The sample is analyzed using a MALDI-TOF mass spectrometer and the PNA probes present in the sample are identified and used to determine a condition(s) of interest associated therewith.

All literature and similar materials cited in this application, including but not limited to patents, patent applications, articles, books and treatises, regardless of the format of such literature or similar material, are expressly incorporated by reference herein in their entirety for any and all purposes.

While the present teachings are described in conjunction with various embodiments, it is not intended that the present teachings be limited to such embodiments. On the contrary, the present teachings encompass various alternatives, modifications and equivalents, as will be appreciated by those of skill in the art. Thus, the invention as contemplated by applicants extends to all functionally equivalent structures, methods and uses, such as are within the scope of the appended claims.

Moreover, in the following claims it should be understood that the order of steps or order for performing certain actions (e.g. mixing of reactants) is immaterial so long as the present teachings remain operable. Unless expressly stated otherwise or where performing the steps of a claim in a certain order would be non-operative, the steps and/or substeps of the following claims can be executed in any order. Moreover, two or more steps or actions can be conducted simultaneously.

1. A method comprising: 1) contacting microorganisms or cells of a sample with one or more hybridization probes capable of determining a condition of interest within said microorganisms or cells for a period of time sufficient for said hybridization probes to sequence specifically hybridize to their respective target sequences associated with said condition of interest within said microorganisms or cells; 2) washing said microorganisms or cells to remove excess hybridization probes; 3) lysing said microorganisms or cells to produce a cell lysate; and 4) analyzing said cell lysate by MS to identify one or more hybridization probes contained in said cell lysate.

2. The method of claim 1 further comprising, determining one or more conditions of interest associated with said microorganisms or cells where said one or more conditions of interest correlate with the identity of said one or more hybridization probes identified in step 4) of the method.

3. The method of claim 2 further comprising, making a diagnostic determination based upon the conditions of interest so determined.

4. The method of claim 3 further comprising, making a treatment recommendation for a subject from whom the sample was obtained based upon said diagnostic determination.

5. A method comprising: 1) contacting microorganisms or cells of a sample with one or more hybridization probes capable of determining a condition of interest within said microorganisms or cells for a period of time sufficient for said hybridization probes to sequence specifically hybridize to their respective target sequences and thereby produce a probe/target complexes associated with said condition of interest within said microorganisms or cells; 2) washing said microorganisms or cells to remove excess hybridization probes; 3) treating said microorganisms or cells with heat and/or other denaturing conditions for a period of time sufficient to thereby cause said probe/target complexes to denature and the denatured probes to diffuse outside of the intact cells/microorganisms; 4) recovering said denatured probes that exist outside of said intact cells/microorganisms; and 5) analyzing said recovered probes by MS to identify one or more of said recovered probes.

6. The method of claim 5 further comprising, determining one or more conditions of interest associated with said microorganisms or cells where said one or more conditions of interest correlate with the identity of said one or more recovered probes identified in step 5) of the method.

7. The method of claim 6 further comprising, making a diagnostic determination based upon the conditions of interest so determined.

8. The method of claim 7 further comprising, making a treatment recommendation for a subject from whom the sample was obtained based upon said diagnostic determination.
